(19) United States
(12) Patent Application Publication
(10) Pub. No.: US 2004/0029219 A1
(43) Pub. Date: Feb. 12, 2004

(54) Candida hemolysin-like proteins

(76) Inventors: David R. Soll, Iowa City, IA (US); Salil A. Lachke, Iowa-City, IA (US); Thyagarajan Srikantha, Coralville, IA (US)

Correspondence Address: FOLEY AND LARDNER, SUITE 500, 3000 K STREET NW, WASHINGTON, DC, 20007, US

(21) Appl. No.: 10/169,103

(22) PCT Filed: Jan. 11, 2001
(86) PCT No.: PCT/US01/00815

(51) Int. Cl. C12P 21/02; C12N 1/18; C12N 15/74; C07K 14/40; C07H 21/04

(52) U.S. Cl. 435/69.1; 435/254.22; 435/320.1; 530/350; 536/23.7

(57) ABSTRACT

Candida yeast such as C. glabrata, express a hemolysin-like protein (HLP) that is structural similarity to hemolysins in other pathogens. HLP polypeptides are antigenic and are useful as vaccine components to raise a protective immune response against Candida. HLP expression is regulated by phenotype switching in C glabrata and coincides with the hemolytic activity of the phenotype.

### BACKGROUND OF THE INVENTION

Candida albicans and C. glabrata Present a Growing Risk to Public Health

The yeast Candida species can exist both as a non-virulent colonizer (commensal) and as a pathogen. Candidiasis has become increasingly widespread during the last two decades, with hospitalized and immunocompromised patients at greatest risk, and has become the sixth most common form of pathogenic infection. Systemic Candida infections may be lethal, with a mortality rate of 50% in adults and up to 65% in infants. Colombo et al. (1999); Pacheco-Rios et al. (1997). Reviewed in Pfaller (1996). The risk of death from systemic infection most strongly correlates with the time between the first detected infection and the onset of antifungal treatment. Pacheco-Rios et al. (1997).

Candida albicans accounts for 50-70% of candidiasis cases, although a progressively larger proportion of cases are caused by non-albicans species. C. glabrata (formerly Torulopsis glabrata) has emerged as one of the three most common Candida species colonizing humans (Fidel et al. (1999); Hazen et al. (1995)) and it now represents the second most common Candida species causing blood stream infections (Pfaller et al. (1996)) and one of the most prevalent species responsible for yeast vaginitis (Sobel et al. (1996); Spinillo et al. (1995)). A dramatic increase in the carriage of C. glabrata has also been demonstrated in dentate individuals over 80 years of age, and the proportion of elderly individuals with dentures carrying C. glabrata in one study was found to be greater than 50%. Lockhart et al. (1999). What is most worrisome about the recent emergence of C. glabrata, as a major Candida commensal and pathogen, is that it is naturally resistant to azole drug therapy. Blaschke-Hellmessen et al. (1996); Fortun et al. (1997); Hitchcock et al. (1993); Marichal et al. (1997).

Candida Pathogenicity is Enhanced by High Frequency Phenotypic Changes

The pathogenic success of Candida depends in part upon phenotypic plasticity. C. albicans, for example, exhibits a bud-hypha phenotype transition, which occurs en masse in response to various stimuli, and provides C. albicans with the capacity to penetrate tissue and to disseminate. Odds (1997). C. albicans also undergoes spontaneous, reversible, high frequency switching of phenotypes, which usually does not occur en masse. In C. albicans strain WO-1, switching occurs reversibly between a phenotype characterized by white colonies and a phenotype characterized by opaque colonies. Soll (1992). In C. albicans, switching has been demonstrated to occur at higher frequencies in isolates from deep versus superficial mycoses (Jones et al. (1994)), at higher frequencies in infecting versus commensal isolates from the oral cavity (Hellstein et al. (1993)), within sites of infection (Soll et al. (1987); Soll et al. (1988)) and within sites of commensalism (Soll (1992)). Switching has also been demonstrated to regulate virulence in animal models. Kvaal et al. (1999).

High frequency phenotypic switching in C. albicans involves the coordinated regulation of phase-specific genes. The gene products of several of these genes facilitate pathogenesis (Soll (1992); Soll (1996)), and they include secreted aspartyl proteinases (Hube et al. (1994); Morrow et al. (1992); Morrow et al. (1993); White et al. (1993)) and drug resistance proteins (Balan et al. (1997)). No single phenotypic trait has been found to be solely responsible for pathogenesis. Switching results in antigenic variability on the yeast cell surface; one cell surface antigen has been identified as specific to the opaque phenotype. Reviewed in Soll (1992). Accordingly, switching provides a mechanism to enhance pathogenesis by generating phenotypic plasticity.

With the emergence of drug-resistant Candida strains and a growing population of immunocompromised individuals, there is an increasing need to find new methods of treating candidiasis. With this goal in mind, there is a need to identify proteins that either are associated with virulent Candida phenotypes or that are themselves pathogenic factors. Antibodies against these proteins also are expected to inhibit the pathogenic activity of these proteins, thus contributing to amelioration of the symptoms of candidiasis and preferably to a decrease in mortality associated with systemic infection. For example, vaccines may be directed against proteins associated preferentially with pathogenic phenotypes.

### SUMMARY OF THE INVENTION

The present invention addresses these needs by providing, from Candida yeast such as C. glabrata, a hemolysin-like protein (HLP) and its encoding polynucleotide. The inventors' discovery of an HLP in C. glabrata led to their identifying a similar C. albicans HLP and encoding gene. The inventors have found that C. glabrata, like C. albicans, undergoes phenotype switching, which regulates the expression of the C. glabrata HLP. HLP expression coincides with hemolytic activity, and it is expected that a phenotype expressing an HLP will have virulent properties conferred by the hemolytic activity of that HLP. The HLP of the invention is expected to exhibit hemolytic activity also because of structural similarity to partial (α-type) or complete (β-type) hemolysins identified in a number of other pathogens.

HLP polypeptides of the invention are antigenic and are useful as vaccine components to raise a protective immune response against Candida. Further, antibodies directed against a Candida HLP, or an antigenic fragment thereof, are useful in indicating the presence of a virulent phenotype. The antibodies also can be used to protect against or ameliorate candidiasis associated with a virulent Candida phenotype. In one embodiment, antibodies raised by an HLP protein, or an antigenic fragment thereof, are capable of inhibiting hemolytic activity of an HLP.

Accordingly, the invention provides a vaccine, comprising:

(i) an antigenic polypeptide comprising 10 contiguous amino acid residues of either of the proteins shown in SEQ ID NOS:2 or 4; or

(ii) a polynucleotide encoding an antigenic polypeptide comprising 10 contiguous amino acid residues of either of the proteins shown in SEQ ID NOS:2 or 4, wherein the polynucleotide is operably linked to a promoter capable of driving transcription in a host cell; and

(iii) a pharmaceutically acceptable adjuvant or carrier vehicle.

The polypeptide component of this vaccine alternately may comprise 12 contiguous amino acids of either of the proteins shown in SEQ ID NOS:2 or 4. Alternately, it may comprise 30 or 40 contiguous amino acids of either of the proteins shown in SEQ ID NOS:2 or 4. In one embodiment, the carrier vehicle is a protein that is covalently conjugated to the polypeptide. In another embodiment, the vaccine comprises either of the proteins shown in SEQ ID NOS:2 or 4.

The vaccine alternately may comprise a polynucleotide encoding a polypeptide comprising 10, 12, 30, or 40 contiguous amino acids of either of the proteins shown in SEQ ID NOS:2 or 4. In one embodiment, the polynucleotide encodes either of the proteins shown in SEQ ID NOS:2 or 4.

A method of diagnosing a virulent phenotype of Candida comprises exposing a body fluid from an individual suspected of having a virulent phenotype of Candida with a detectably labeled antibody that is capable of binding an HLP of Candida.

A method of treatment of an individual infected with a virulent phenotype of Candida comprises administration of a pharmaceutical composition that comprises an antibody that is capable of binding an HLP of Candida. In one embodiment, a method of inducing an immune response, preferably comprising a cellular immune response, comprises administering to an individual a pharmaceutical composition that comprises either:

(i) an antigenic polypeptide comprising 10 contiguous amino acid residues of either of the proteins shown in SEQ ID NOS:2 or 4; or

(ii) a polynucleotide encoding an antigenic polypeptide comprising 10 contiguous amino acid residues of either of the proteins shown in SEQ ID NOS:2 or 4, wherein the polynucleotide is operably linked to a promoter capable of driving transcription in a host cell; and

(iii) a pharmaceutically acceptable adjuvant or carrier vehicle.

Another embodiment of the invention is an isolated polynucleotide that exhibits greater than about 80% sequence identity to polynucleotide sequence SEQ ID NO:1, or a polynucleotide having the sequence shown in SEQ ID NO:1, or a polynucleotide encoding a polypeptide having the sequence shown in SEQ ID NO:2. Polypeptides encoded by the polynucleotides are encompassed by the invention, including a polypeptide having the sequence shown in SEQ ID NO:2. Antigenic fragments preferably comprises at least 30 contiguous amino acids of the protein shown in SEQ ID NO:2.

BRIEF DESCRIPTION OF THE FIGURES

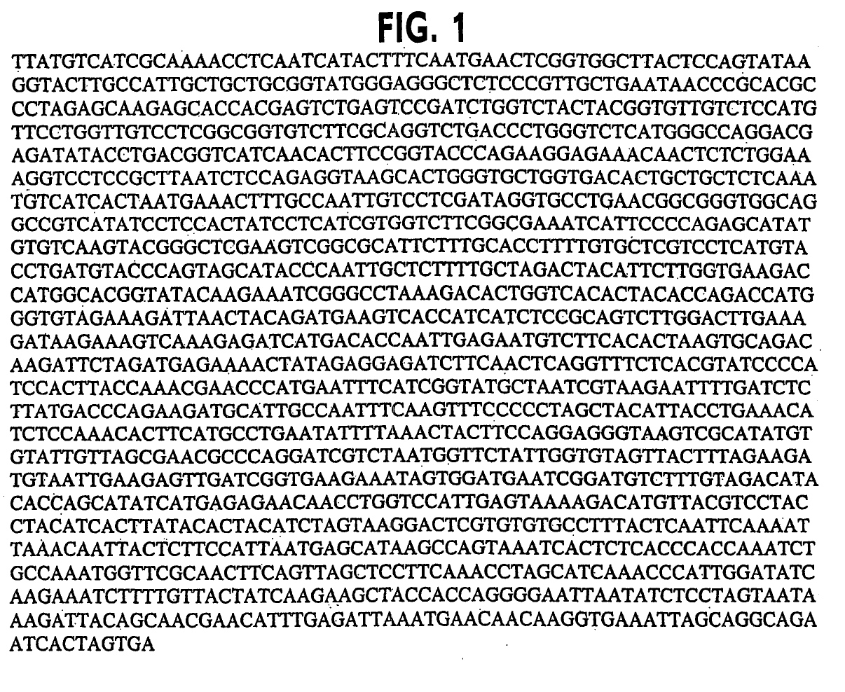

FIG. 1: A cloned C. glabrata genomic DNA fragment that is 1532 nucleotides in length (SEQ ID NO: 1).

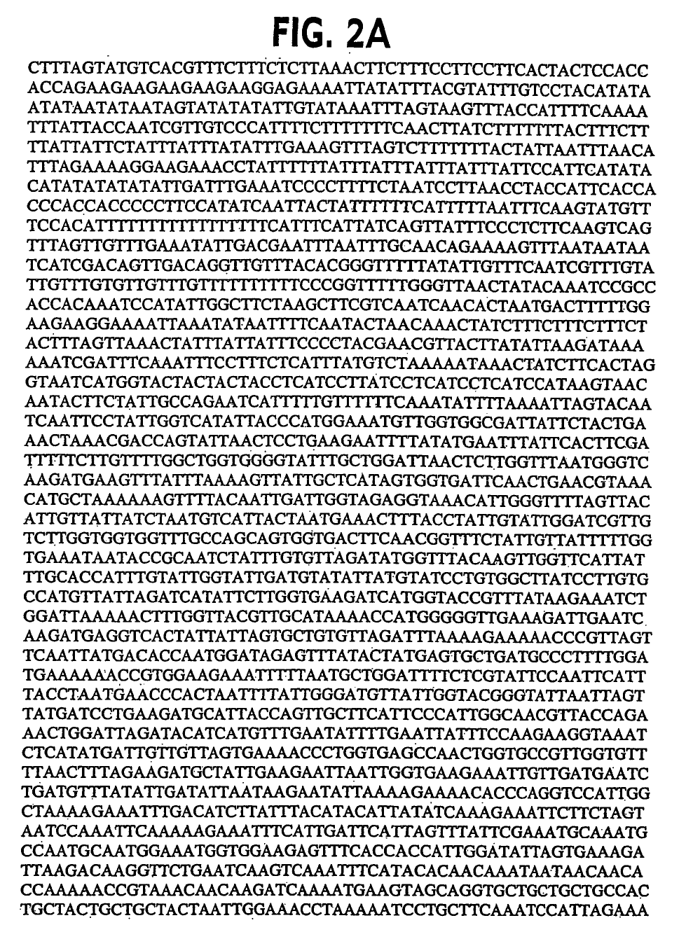

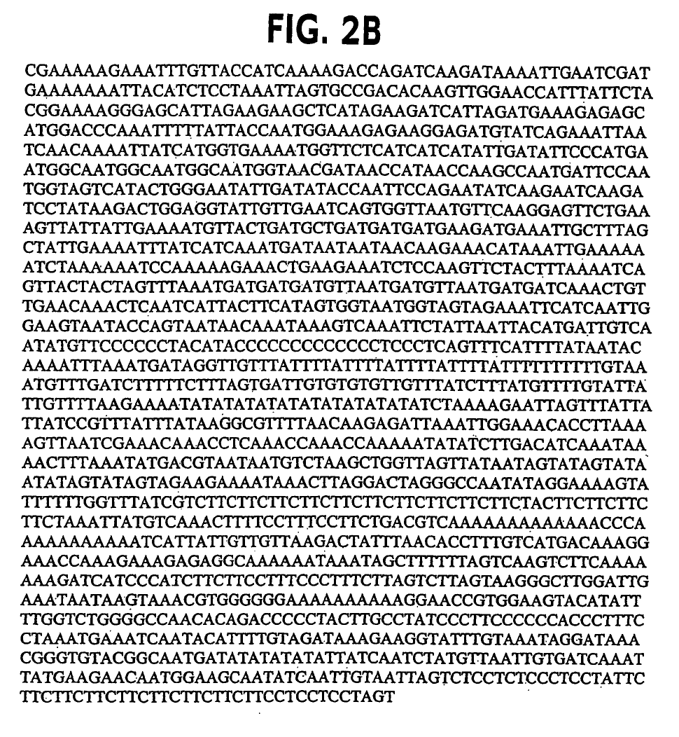

FIG. 2: The polynucleotide sequence of a C. albicans HLP-encoding DNA, including a promoter region, cloned from C. albicans strain WO-1 (SEQ ID NO:3).

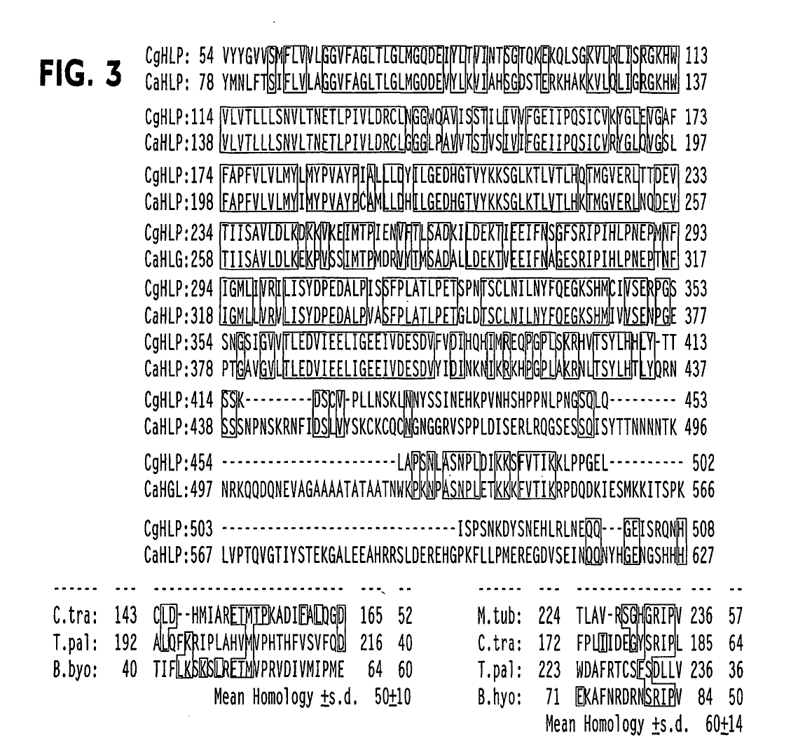

FIG. 3: Comparison of the deduced amino acid sequence of a C. glabrata HLP (“CgHLP”; SEQ ID NO:2) and C. albicans HLP (“CaHLP”; SEQ ID NO:4).

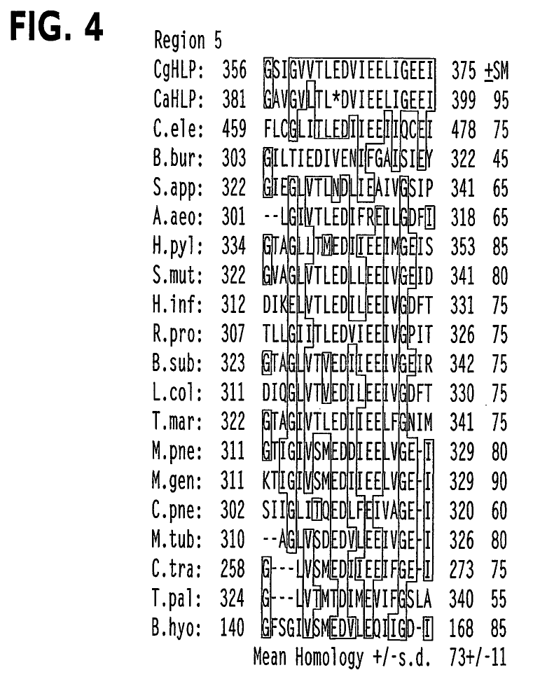

FIG. 4 : Comparison of five regions with high similarity in both position and arrangements of amino acids among a C. glabrata and C. albicans HLP and hemolysins from seventeen bacteria and Caenorizabditis elegans.

### DETAILED DESCRIPTION OF THE PREFERRED EMBODIMENTS

The inventors have identified polynucleotides and the encoded polypeptides of Candida hemolysin-like proteins (HLPs) and presently disclose HLPs from C. albicans and C. glabrata. The inventors have further discovered a novel phenotype switching in C. glabrata. C. glabrata reversibly switches among white (Wh), light brown (LB) and dark brown (DB) colony phenotypes. Switching in C. glabrata, as in C. albicans, is believed to represent a general strategy for the combinatorial expression of genes encoding proteins involved in virulence. In particular, the C. glabrata HLP is expressed preferentially in different phenotypes, and, by analogy with C. albicans, the phenotype expressing the HLP at high levels is expected to express other virulence factors at high levels.

Hemolytic activity will play a particularly important role in pathogenesis, especially in systemic infection by Candida, where circulating red blood cells are exposed directly to the pathogens. It has been postulated that Candida acquires iron that is liberated from lysed red blood cells of the infected host. Manns et al. (1994). Further, in various ways beyond the outright loss of red blood cells, hemolysis impairs the health of the host and contributes to mortality from systemic infection. Reviewed in Maslow et al. (1999); Traub et al. (1996); May et al. (1996); Hacker et al. (1983). Accordingly, inhibition of hemolytic activity will be valuable in the treatment of candidiasis. In one embodiment of the invention, antibodies against an HLP inhibit hemolytic activity.

Regardless of whether other virulence factors are produced in combination with HLP, the association of HLP with hemolytic activity is, itself, sufficient to identify a phenotype that expresses high levels of an HLP as a virulent phenotype. With this in mind, any Candida phenotype that expresses an HLP is defined as a “virulent phenotype,” for the purposes of this invention.

Similarity of C. albicans and C. glabrata HLP with Known Hemolysins

HLPs of the invention are believed to exhibit hemolytic activity, because phenotypes expressing these HLPs at high levels are hemolytic, and because the HLPs are structurally related to other hemolysins exhibiting either partial (α-type) or complete (β-type) hemolysis identified in a number of other pathogens. Fairweather et al. (1983); Helms et al. (1977); Li et al. (1999); Leao et al. (1995). Electron microscopy suggests that hemolysins lyse cells by forming oligomeric pore complexes through the cellular plasma membrane of the target cell. Nizet et al. (1997). See also Lange et al. (1997).

A polynucleotide encoding an HLP of the invention was found serendipitously during in an attempt to clone another gene in C. glabrata. FIG. 1 shows a cloned C. glabrata genomic DNA fragment that is 1532 nucleotides in length and encodes an HLP with three putative trans-membrane domains. The discovery of a hemolysin-like gene in C. glabrata prompted the inventors to investigate the possible presence of a similar gene in C. albicans. The polynucleotide sequence of an HLP gene in C. albicans is shown in FIG. 2 (SEQ ID NO:2).

Comparison of the deduced amino acid sequence of a C. albicans and C. glabrata HLP revealed an overall 75% sequence identity spanning 351 amino acid residues of the protein. Both the amino-terminal end consisting of 60-85 amino acid residues and carboxy-terminal end consisting of over 400 amino acid residues appear unique to each species. See FIG. 3 (SEQ ID NOS:2 and 4).

Comparison of these two Candida HLPs with known hemolysins revealed similarity among a variety of pathogenic and nonpathogenic bacteria as well as eukaryotes. Gish et al. (1993); Worley et al. (1995). These hemolysins range in size from 65-85 kD. Welch (1995); Tweter (1995). In particular, comparison with data bases revealed five regions with high similarity in both position and arrangements of amino acids among a C. glabrata and C. albicans HLP and hemolysins from seventeen pathogenic microorganisms and Caenorhabditis elegans. FIG. 4. The mean percent identity of region 1 of the deduced C. glabrata protein sequence and hemolysins from 16 unrelated organisms was 72±12%; percent identity ranged between 42 and 92%, where “percent identity” refers to the similarity between sequences based on the BLASTX-BEAUTY sequence alignment algorithm, using the indicated C. glabrata sequence as a reference sequence. The mean percent identities of regions 2, 3, 4 and 5 were 59±16%, 50±10%, 60±14% and 73±11%, respectively. Furthermore, the relative positions of all five regions in the deduced partial C. glabrata protein were similar to those in the majority of hemolysins of other organisms. The deduced primary C. glabrata HLP sequence of 508 amino acids exhibits 75% identity to the corresponding region of the S. cerevisiae YOL060c gene product.

A C. albicans hemolytic factor has recently been identified by Wanatabe et al. (1999). This factor has been partially characterized and is primarily a polysaccharide, consisting of 95% carbohydrate by weight. It co-migrates with Blue Dextran on a Sephacryl S-100 column, indicating that it has an apparent molecular weight of at least 200 kDa, and it is a mannan. It is not believed that the mannan described by Wanatabe et al. is structurally related to the C. albicans HLP shown in SEQ ID NO:4.

HLP Polypeptides of the Invention

The phrase “hemolysin-like protein” denotes a polypeptide having the amino acid sequence shown in SEQ ID NO:2, which is a C. glabrata HLP, or having the amino acid sequence shown in SEQ ID NO:4, which is a C. albicans HLP. Naturally occurring and synthetically produced variants of these two proteins also are embraced by “hemolysin-like protein.” “Variants” encompass, for example, naturally occurring HLPs, including HLPs from other Candida species and allelic variations of these proteins. Among “variants” in this context also are fragments of the aforementioned proteins that are antigenic when administered with an adjuvant or carrier vehicle. Preferably, fragments are contiguous stretches of amino acids of the proteins of SEQ ID NOS:2 and 4 that are 5, 10, 12, 15, 20, 30, or 40 amino acids in length.

In addition, “variants” further include polypeptides that have a modified amino acid sequence from the aforementioned polypeptides. The skilled artisan will recognize that structure ultimately defines function, and that variants bearing the closest structural relationship to HLPs shown in SEQ ID NOS:2 and 4 are most likely to preserve biological function and antigenicity. Sequence modifications include amino acid substitutions, insertions, and deletions. Amino acid insertions and deletions may be made in the interior of the protein sequence, as well as at the amino and carboxyl termini. Guidance in determining which and how many such sequence modifications may be made without abolishing biological or antigenic activity may be found using computer programs well known in the art, for example, DNASTAR software (DNASTAR Inc., 1801 Univerity Ave., Madison, Wis. 53705).

Substitutions preferably are conservative, that is, one amino acid is replaced with one of similar shape and charge. Conservative substitutions are well known in the art and include, for example, the changes of: alanine to serine; arginine to lysine; asparigine to glutamine or histidine; aspartate to glutamate; cysteine to serine; glutamine to asparigine; glutamate to aspartate; glycine to proline; histidine to asparigine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The sequence of variants preferably will have an 80% identity to the full length proteins shown in SEQ ID NOS:2 and 4. Again, “percent identity” is determined by the BLASTX-BEAUTY sequence algorithm, where the test sequence is aligned and compared to either sequences set forth in SEQ ID NOS:2 and 4. More preferably, variants will have at least about 85% identity to these sequences. Even more preferably, the percent identity will be at least about 90%, and most preferably, the percent identity will be at least about 95%, or even 98%. Likewise, variants of fragments of the proteins of SEQ ID NOS:2 and 4 will be useful for the invention, for instance, as antigenic fragments. Such variants will have at least about 85% identity to fragments of the proteins of SEQ ID NOS:2 and 4. Even more preferably, the percent identity will be at least about 90%, and most preferably, the percent identity will be at least about 95%, or even 98%. Preferably, antigenic fragments will be at least 30 amino acids in length.

Additional polypeptide sequences or other moieties, such as covalently attached, detectable tags, may be added to the proteins of the invention. Additional polypeptide sequences may fused to either the amino or carboxyl termini of the polypeptides of the invention, and they may be useful, for example, in assisting the expression, purification, and/or detection proteins of the invention. For example, these various sequences include those well known in the art that are useful in purification of recombinantly expressed proteins. A preferred fusion protein comprises a “His tag” sequence, which facilitates purification of the recombinantly expressed protein. A preferred system is the TALONS nondenaturing protein purification kit for purifying 6xHis-tagged proteins under native conditions (CLONTECH, Palo Alto, Calif.).

“Isolated” polypeptides are not in a naturally occurring form and/or have been purified to remove at least some portion of cellular or non-cellular molecules with which the proteins are naturally associated. The present HLPs may be isolated by the methodology described by Herbelin et al. (1995) for the purification of hemolysins, for example.

Assays for Hemolytic Activity

An HLP is expected to exhibit hemolytic activity, as set forth above. In one embodiment, an HLP variant exhibits hemolytic activity. It is expected that a hemolytically active HLP variant will include some or all of the structural elements that are conserved among known hemloysins, indicated in FIG. 4. The HLPs of the invention bear closest resemblance to hemolysins that exhibit partial (α-type) or complete (β-type) hemolysis. Fairweather et al. (1983); Helms et al. (1977); Li et al. (1999); Leao et al. (1995).

Hemolytic activity can be tested by any of several routine assays well known in the art. These assays include inspection of cells exposed to hemolysins by light microscopy, release of detectable cytoplasmic proteins, such as hemoglobin or enzymes, Trypan blue exclusion, or Evans blue-albumin flux. Gibson et al. (1999); Thelestam et al. (1994). Additional assays include a hemolytic plate assay or cell free broth assay as described in Parveen (1992). Alternately, a fluorometric assay, using erythrocyte ghosts, may be used, as described in Serra et al. (1992).

These assays can be used to test the ability of compounds to inhibit the hemolytic activity of an HLP or an HLP variant. High throughput assays, designed around one of the assays described above, for example, can simultaneously test a plurality of candidate compounds. Antibodies will be screened using a hemolytic assay to identify those antibodies with inhibitory activity, which will allow the identification of potentially therapeutically useful antibodies. This screening method can also be applied to other candidate molecules, such as drugs, to identify those with inhibitory activity. Such drugs are expected to be useful as components of a pharmaceutical composition for treatment of candidiasis.

HLP Polynucleotides of the Invention

A polynucleotide of the invention encodes any polypeptide of the invention, and includes all the possible combinations of codons that encode particular amino acids. Polynucleotides of the invention, in addition to encoding polypeptides, may be useful as oligonucleotide probes for the identification of other HLP polynucleotides, as set forth in the Examples below. A polynucleotide comprises at least 5 nucleotides of a nucleic acid (RNA, DNA, a combination thereof, or analogues thereof), provided by any means, such as synthetic purification. Polynucleotides having at least about 10 nucleotides are preferred, and polynucleotides having at least about 20 nucleotides are more preferred. The nucleotide sequence of an HLP gene fragment shown in FIG. 1 (SEQ ID NO: 1) has been deposited in the National Center for Biological Information under accession number AF196836.

Methods of Isolating Polynucleotides of the Invention

Identification of polynucleotides encoding HLP in C. glabrata and C. albicans will allow the skilled artisan to identify structurally related ploynucleotides encoding variants of these polynucleotides, other polynucleotides from other Candida species. Methods suitable for identification of related polynucleotides are well known in the art.

Three such approaches are described in the Examples, below. They are (1) a PCR-based strategy using degenerate primers, (2) the use of a non-hemolytic Saccharomyces cerevisiae system as a screen for genes for Candida hemolytic activity, and (3) a search for HLP homologs in Candida by “mining” nucleic acid and protein databases, followed by functional and structural characterization of the suspect genes and gene products.

Vectors and Host Cells of the Invention

The invention also provides vectors and host cells that comprise the polynucleotides of the invention. Host cells include any eukaryotic, prokaryotic, or other cell that is suitable for propagating and/or expressing an isolated nucleic acid that is introduced into the host cell by any suitable means known in the art. The cell can be part of a tissue or organism, isolated in culture or in any other suitable form. Vectors include any nucleic acid compound used for introducing exogenous nucleic acid into host cells. A vector comprises a nucleotide sequence which may encode one or more polypeptide molecules. Plasmids, cosmids, viruses, and bacteriophages, in a natural state or which have undergone recombinant engineering, are non-limiting examples of commonly used vectors to provide recombinant vectors comprising at least one desired isolated nucleic acid molecule. Reviewed in Sambrook et al. (1989) and Ausubel et al. (1989). Because of the similarity of the HLP proteins to partial (α-type) or complete (β-type) hemolysis, it is expected that they will be functional hemolysins in secreted form. Accordingly, preferred expression vectors comprise any of the well-characterized secretion signals.

A host cell of the invention strain may be chosen for its ability to post-translationally modify the expressed recombinant protein. Such modifications of the polypeptide include, for example, glycosylation and acylation. Post-translational processing which cleaves a nascent form of the protein may also be important for correct secretion, folding and/or function. Different host cells may be chosen to ensure the correct modification and processing of the recombinantly expressed protein.

“Isolated” polynucleotides are removed from their native or naturally occurring environment. For example, recombinant polynucleotides within vectors are considered isolated for the purposes of the present invention. Isolated polynucleotides include in vivo or in vitro RNA transcripts of the polynucleotides of the invention. Isolated polynucleotides further include synthetically produced molecules, where the nucleic acid in other than a naturally occurring form. Isolated polynucleotides include genomic DNA that has been removed from the chromosome in which it occurs naturally.

Antibodies and Vaccines of the Invention

Candida HLPs and antigenic fragments thereof will be useful as components of a vaccine. In one embodiment, a vaccine comprises HLP from C. albicans or C. glabrata, or antigenic fragments thereof. Particularly useful vaccines will raise antibodies that block or inhibit the hemolytic activity of the protein. Such antibodies can be screened in vitro, using the various hemolysis assays described above.

Protection against pathogens by vaccination against a hemolysin antigen has met with considerable success. Furesz et al. (1997); Haga et al. (1997); Byrd et al. (1997); U.S. Pat. No. 6,007,825; U.S. Pat. No. 5,731,151. In some instances, the hemolytic activity of a hemolysin was blocked by polyclonal antibodies. For example, Lange et al. (1997).

In general, the preparation of polyclonal and monoclonal antibodies, as well as hybridomas capable of producing the desired antibody, are well known in the art. For example, Campbell (1984); St. Groth et al. (1980); Kohler et al. (1975); Kozbor et al. (1983); Cole et al. (1985).

i) Polyclonal Antibodies

Polyclonal antiserum, containing antibodies to heterogenous epitopes of a single protein, can be prepared by immunizing suitable animals with the expressed protein described above, which can be unmodified or modified, as known in the art, to enhance immunogenicity. Immunization methods include subcutaneous or intraperitoneal injection of the polypeptide.

The protein immunogen may be modified or administered in an adjuvant in order to increase the protein's antigenicity. Methods of increasing the antigenicity of a protein are well known in the art and include, but are not limited to, the inclusion of an adjuvant during immunization. Adjuvants include Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

HLP antigens alternately may be conjugated to a carrier vehicle, or “immunocarrier,” to improve the interaction between T and B cells for the induction of an immune response against the antigen. This is particularly preferable for the production of vaccines in immunocompromised individuals, who also will be those predisposed to candidiasis. Preferred carrier vehicles are proteins that are covalently conjugated to a HLP polypeptide of the invention. Proteins useful as carrier vehicles are well known in the art and include tetanus toxoid, diphtheria toxoid, P. aeruginosa exotoxin A, and variants thereof, as described, for example, in Fattom et al. (1993).

Effective polyclonal antibody production is affected by many factors related both to the antigen and to the host species. For example, small molecules tend to be less immunogenic than other and may require the use of carriers and/or adjuvant. In addition, host animal response may vary with site of inoculation. Both inadequate or excessive doses of antigen may result in low titer antisera. In general, however, small doses (high ng to low μg levels) of antigen administered at multiple intradermal sites appears to be most reliable. Host animals may include but are not limited to rabbits, mice, and rats, to name but a few. An effective immunization protocol for rabbits can be found in Vaitukaitis et al. (1971). Affinity of the antisera for the antigen may be determined by preparing competitive binding curves, as described, for example, by Fisher(1980).

ii) Monoclonal Antibodies

Monoclonal antibodies (MAbs) may be obtained by any technique that provides for the production of antibody molecules by continuous cell lines in culture or in vivo. MAbs may be produced by making hybridomas, which are immortalized cells capable of secreting a specific monoclonal antibody. Monoclonal antibodies to any of the proteins, peptides and epitopes thereof described herein can be prepared from murine hybridomas according to the classical method of Kohler et al. (1975) and U.S. Pat. No. 4,376,110, or modifications of the methods thereof. Such modifications include the human B-cell hybridoma technique (Kosbor et al. (1983)) and the EBV-hybridoma technique (Cole et al. (1985)).

iii) Antibody Derivatives and Fragments

Fragments or derivatives of antibodies include any portion of the antibody which is capable of binding the target antigen, or a specific portion thereof. Antibody fragments specifically include F(ab′)₂, Fab, Fab′ and Fv fragments. They may be made by conventional recombinant DNA techniques or, using the classical method, by proteolytic digestion with papain or pepsin. See Current Protocols in Immunology, chapter 2, Coligan et al., eds., John Wiley & Sons (1991-92). Other antibody derivatives include single chain antibodies. U.S. Pat. No. 4,946,778; Bird (1988); Huston et al. (1988). Single chain antibodies are formed by linking the heavy and light chain fragments of the Fv region via an amino acid bridge, resulting in a single chain FV (SCFv).

Derivatives also include “chimeric antibodies.” Morrison et al. (1984); Neuberger et al. (1984); Takeda et al. (1985). These chimeras are made by splicing the DNA encoding a mouse antibody molecule of appropriate specificity with, for instance, DNA encoding a human antibody molecule of appropriate specificity. Thus, a chimeric antibody is a molecule in which different portions are derived from different animal species, such as those having a variable region derived from a murine mAb and a human immunoglobulin constant region. These are also known sometimes as “humanized” antibodies and they offer the added advantage of at least partial shielding from the human immune system. They are, therefore, particularly useful in therapeutic in vivo applications.

iv) Labeled Antibodies

The present invention further provides the above-described antibodies in detectably labeled form. Antibodies can be detectably labeled through the use of radioisotopes, affinity labels (such as biotin, avidin, etc.), enzymatic labels (such as horseradish peroxidase, alkaline phosphatase, etc.) fluorescent labels (such as FITC or rhodamine, etc.), paramagnetic atoms, etc. Labeling procedures are well known in the art, for example see Sternberger et al. (1970); Bayer et al. (1979); Engval et al. (1972). The labeled antibodies of the present invention can be used for in vitro, in vivo, and in situ diagnostic assays.

v) Immobilized Antibodies

The foregoing antibodies also may be immobilized on a solid support. Examples of such solid supports include plastics such as polycarbonate, complex carbohydrates such as agarose and sepharose, acrylic resins and such as polyacrylamide and latex beads. Techniques for coupling antibodies to such solid supports are well known in the art. Weir et al. (1986). The immobilized antibodies of the present invention can be used for in vitro, in vivo, and in situ assays as well as for immunoaffinity purification of the proteins of the present invention.

vi) DNA Vaccines

DNA-based immunization refers to the induction of an immune response to an antigen expressed in vivo from a gene introduced into the animal. This method offers two major advantages over classical vaccination in which some form of the antigen itself is administered. First, the synthesis of antigen in a self-cell mimics in certain respects an infection and thus induces a complete immune response but carries absolutely no risk of infection. Second, foreign gene expression may continue for a sufficient length of time to induce strong and sustained immune responses without boost. See U.S. Pat. No. 5,780,448, for example.

Genes have been introduced directly into animals by using live viral vectors containing particular sequences from an adenovirus, an adeno-associated virus, or a retrovirus genome. The viral sequences allow the appropriate processing and packaging of a gene into a virion, which can be introduced to animals through invasive or non-invasive infection. Naked DNA transfects relatively efficiently if injected into skeletal muscle. Wolff et al. (1990). Alternately, plasmid DNA may be coated onto the surface of small gold particles and introduced into the skin by a helium-driven particle accelerator, or “gene-gun.” Pecorino et al. (1992). DNA has also been introduced into animal cells by liposome-mediated gene transfer. DNA-liposome complexes, usually containing a mixture of cationic and neutral lipids, are injected intravenously or into various tissues or instilled into the respiratory passages. Nabel et al. (1992).

Several mammalian animal models of DNA-based immunization against specific viral, bacterial or parasitic diseases have been reported. In most of these studies, a full-range of immune responses, including antibody production and a cytotoxic T lymphocyte response, was obtained. See U.S. Pat. No. 5,780,448, for example.

Diagnostic Methods of the Invention

A variety of protocols useful for detecting and measuring the presence of HLPs of the invention in body fluids or tissue and cell extracts, using either polyclonal or monoclonal antibodies specific for the protein, are known in the art. Examples include enzyme-linked immunosorbent assay (ELISA), radioimmunoassay (RIA) and fluorescent activated cell sorting (FACS). A two-site, monoclonal-based immunoassay utilizing monoclonal antibodies reactive to two non-interfering epitopes may be employed. Well known competitive binding techniques may also be employed. See, e.g., Hampton et al. (1990); Coligan et al. (1997 and periodic supplements); and Maddox et al. (1983).

The immunoassays of this invention can be embodied in test kits which comprise HLP proteins of the invention or HLP-specific antibodies. Such test kits can be in solid phase or liquid phase formats, and they can be based on immunohistochemical assays, ELISAs, particle assays, radiometric or fluorometric assays, using, for example, avidin/biotin technology.

Therapeutic Methods of the Invention

A vaccine of the present invention can be administered to a subject who then acts as a source for globulin, produced in response to challenge from the specific vaccine (“hyperimmune globulin”), that contains antibodies directed against an HLP of the invention. U.S. Pat. No. 5,770,208. A subject thus treated would donate plasma from which hyperimmune globulin would then be obtained, via conventional plasma-fractionation methodology, and administered to another subject in order to impart resistance against or to treat Candida infection. Hyperimmune globulins according to the invention are particularly useful for immune-compromised individuals, for individuals undergoing invasive procedures or where time does not permit the individual to produce his own antibodies in response to vaccination.

A method of preparing an immunotherapeutic agent against Candida infection comprises steps of immunizing subjects with a composition according to the invention, collecting plasma from the immunized subjects, and harvesting a hyperimmune globulin that contains antibodies directed against Candida from the collected plasma. The hyperimmune globulin contains antibodies directed against the HLPs of the invention. An immunotherapy method comprises a step of administering this hyperimmune globulin to a subject.

Hyperimmune globulins would preferably be administered in the presence of known compounds that are useful for treating Candida. Amphotericin B is one such compound. Fluconazole has been shown to be an effective and safe alternative in non-neutropenic patients. 5-Fluorocytosine has been used in combination with amphotericin B in the treatment of deep-seated infections. Liposomal formulations of amphotericin B and other antifungal drugs may be used. Verduyn Lunel et al. (1999).

The present invention, thus generally described, will be understood more readily by reference to the following examples, which are provided by way of illustration and are not intended to be limiting of the present invention.

### EXAMPLES

### Example 1

### C. glabrata HLP Expression is Associated with Phenotype Switching

Expression of C. glabrata HLP is regulated by switching, with the order of transcript levels of the various phenotypes DB>LB>Wh. To verify that the levels of C. glabrata HLP transcripts were in fact selectively regulated in Wh, LB, and DB, the transcript levels of additional control genes were analyzed. Transcript levels of various constitutively expressed genes were similar in Wh, LB and DB cells, supporting the conclusion that C. glabrata HLP is selectively regulated by switching.

The level of HLP transcript in the three switch phenotypes of C. glabrata was assessed by slot blot analysis. The level of transcript was lowest in Wh cells, intermediate in LB cells and highest in DB cells. Densitometric scans provided relative transcript ratios of 1:20:25 for Wh:LB:DB.

The experiments assessing switching and gene expression were performed on strain 35B11, an oral isolate from a healthy, elderly individual. Lockhart et al. (1999). In order to test whether high frequency switching was a general characteristic of C. glabrata, switching was tested in three additional C. glabrata isolates, 65FLOP, 65TL1 and 75PLI. For each strain, cells from a single colony were first grown in YPD medium containing 1 mM CuSO₄, then plated at low density on YPD agar containing 1 mM CuSO₄ and colony phenotypes assessed after five days of incubation at 25° C. In every case, multiple phenotypes based on colony color (Wh, LB, DB) were observed at frequencies roughly similar to those observed for strain 35B11. Thus, switching is a general characteristic of C. giabrata strains. Experiments summarizing the frequency of switching among white, light brown and dark brown colony phenotypes in C. glabrata and switching in three additional pathogenic isolates of C. glabrata are summarized in Tables I and II.

TABLE 1
Frequency of alternative phenotypes in white, light brown and dark brown
colonies in C. glabrata.

| | | | Frequency | Frequency | | |
|---|---|---|---|---|---|---|
| | | | Dark | Light | Frequency | Frequency |
| Original | | Number | Brown | Brown | White | Sectored |
| Phenotype | Clone | Colonies | Colonies | Colonies | Colonies | Colonies* |
| Light | 1 | 1234 | 3 × 10⁻² | — | 3 × 10⁻³ | 4 × 10⁻¹ |
| Brown | 2 | 1425 | 2 × 10⁻² | — | 6 × 10⁻³ | 4 × 10⁻¹ |
| (LB) | 3 | 1833 | 3 × 10⁻² | — | 7 × 10⁻³ | 3 × 10⁻¹ |
| | 4 | 1223 | 1 × 10⁻² | — | <10⁻³ | 5 × 10⁻¹ |
| | mean | | 2 × 10⁻² | | 4 × 10⁻³ | 1 × 10⁻¹ |
| | (±s.d.) | | (±1 × 10⁻²⁾ | | (±3 × 10⁻³⁾ | (±1 × 10⁻¹⁾ |
| Dark | 1 | 6418 | — | 8 × 10⁻³ | <2 × 10⁻⁴ | 3 × 10⁻¹ |
| Brown | 2 | 2897 | — | 3 × 10⁻⁴ | 1 × 10⁻³ | 1 × 10⁻³ |
| (DB) | 3 | 2719 | — | 9 × 10⁻³ | 4 × 10⁻⁴ | 2 × 10⁻³ |
| | mean | | | 3 × 10⁻³ | 5 × 10⁻⁴ | 2 × 10⁻³ |
| | (±s.d.) | | | (±5 × 10⁻³⁾ | (±5 × 10⁻⁴⁾ | (±1 × 10⁻³⁾ |
| White | 1 | 792 | 4 × 10⁻¹ | 6 × 10⁻² | — | 6 × 10⁻¹ |
| (Wh) | 2 | 725 | 4 × 10⁻¹ | 7 × 10⁻² | — | 6 × 10⁻¹ |
| | 3 | 1509 | 4 × 10⁻² | 5 × 10⁻² | — | 2 × 10⁻¹ |
| | 4 | 938 | 2 × 10⁻² | 3 × 10⁻² | — | 5 × 10⁻² |
| | 5 | 1422 | 5 × 10⁻² | 4 × 10⁻² | — | 9 × 10⁻² |
| | mean | | 2 × 10⁻¹ | 5 × 10⁻² | | 3 × 10⁻¹ |
| | (±s.d.) | | (±2 × 10⁻¹⁾ | (±2 × 10⁻²⁾ | | (±3 × 10⁻¹⁾ |

*In each case the frequencies of sectors exhibiting all alternative phenotypes are combined

TABLE 2
Switching in three additional pathogenic isolates of C. glabrata.

| | | | | | | | Frequency |
|---|---|---|---|---|---|---|---|
| Original | Number | | | | | Variant | |
| Strain | Phenotype | Colonies | Wh | LB | DB | Sectored | Colonies |
| 65FLOP | LB | 3775 | 5.8 × 10⁻³* | — | 5.8 × 10⁻³ | 9%† | 1.2 × 10⁻² |
| 65TL1 | LB | 2330 | 4.0 × 10⁻⁴ | — | 5.4 × 10⁻² | >80%† | 5.5 × 10⁻² |
| 75PL1 | DB | 3290 | 2.4 × 10⁻³ | 6.0 × 10⁻⁴ | — | 0.4%‡ | 3.0 × 10⁻³ |

*White colonies were further subdivided into 8 normal smooth white and 14 conus smooth white.
†In platings of both the 65 FLOP and 65 TL1 cells, the great majority of sectors in LB colonies were DB.
‡In the DB colonies, 6 Wh sectors, 2 LB sectors and 7 very dark brown sectors were observed.

### Example 2

### PCR-based Strategy Using Degenerate Primers to Identify HLP Homologues

Degenerate primers may be designed that are complementary to stretches of DNA encoding highly conserved domains of HLP proteins. Exemplary highly conserved domains include regions 1 and 3 of the C. albicans HLP indicated in FIG. 2.

| | |
|---|---|
| Region 1: | |
| CaHLP-N: | 5′-ATR GTS RTS TTR GGT GAA ATR ATR CC-3′ |
| Region 3: | |
| CaHLP-C1: | 5′-GGD GTC ATS ATN TCN TNR AC-3′ |
| CaHLP-C2: | 5′-CCA TGG ACA DTC CCA GTR GAM-3′ |

Using specific combinations of forward and reverse primers, PCR reactions may performed using either total genomic DNA or total RNA pool from Candida hypha cultures. The amplifications may be performed using either classical Taq polymerase (Life Technologies, Inc, Gaithersburg, Md.) or high fidelity Long PCR kit (Roche Biochemicals Inc., Indianapolis, Ind.). The cycle conditions used will be 92° C. for 1 min, 36° C. for 1 min and 68° C. for 90 sec and 40 cycles. As compared to the classical Taq polymerase, high fidelity long PCR protocol will be more reproducible and will result in consistent amplified products ranging in size from 250 bp to 500 bp. The pool of PCR products will be cloned into pGEM-T easy vector for sequence analysis. The PCR insert library will be screened and sequenced to reveal clones that encode hemolysin-like genes. Once it is confirmed that the insert contains sequences for a putative hemolysin gene, the full-length gene will be cloned using techniques well known in the art. The regulation of the gene by phenotype switching and the function of the gene product can then be determined.

### Example 3

### Using Non-hemolytic Saccharomyces cerevisiae System to Screen for Genes for Candida hemolytic Activity

S. cerevisiae has been used extensively to identify Candida genes either based on functional complementation of homologous genes or functional induction of new activities associated with heterologous non-homologous genes. Boone et al. (1991); Gillum et al. (1984); Rosenbluh et al. (1985); Fu et al. (1998); Gaur et al. (1997).

The main principle of functional complementation is to identify Candida HLP polynucleotides by gain of function analysis of cells transformed by either genomic or cDNA libraries. Two research groups have recently demonstrated the power of this experimental strategy. Two genes, ALS1 and ALA1, whose products are involved in adherence of C. albicans to host cells, were identified by introducing the C. albicans genomic library into a S. cerevisiae strain and assaying transformants for adherence to endothelial cells (Fu et al. (1998)) or to magnetic beads coated with extracellular matrix proteins (ECMs) (Gaur et al. (1997)). We describe below the strategy for the isolation of genes that encode hemolytic activity of C. albicans.

A genomic library of Candida will be constructed in a 2μ-based S. cerevisiae shuttle vector (PEMBLYe30) with Leu2 as a selectable marker and will be used in the study. A 2μ-based genomic library was chosen rather than a centromere based genomic library because the former system will exhibit greater sensitivity for identifying hemolytic factor due to higher plasmid copy number per cell resulting in production of higher levels of hemolytic activity.

As the first step in this strategy, a Leu2⁻ S. cerevisiae strain will be transformed with a Candida genomic library using lithium acetate transformation protocol. Schiestl et al. (1989). Following transformation, cells will be spread on selective SD agar plates without leucine. After 4 days of growth at 30° C., the total number of transformants will be scored. Approximately 10,000 to 25,000 transformants representing 6 to 8 genomic equivalents (considering the average size of DNA inserts to be 5 to 7 Kb) will be included in the hemolytic activity screen. The individual transformants will be picked and collected in 20 to 25 pools, each pool consisting of approximately 1000 clones. From each pool, approximately 1000 individual cells will be spread on blood agar plates containing human blood (Manns et al., 1994). The plates will be incubated at 30° C. for 3 to 4 days and periodically monitored for hemolytic zones surrounding the colonies. As a control for background, transformants containing the parental plasmid pEMBLYe30 will also be monitored for hemolytic activity. Those transformants exhibiting a zone of hemolytic activity will be selected for further analysis. The plasmid DNA containing Candida putative genes for hemolytic activity will be isolated from the select S. cerevisiae transformants and transformed into E. coli. Both the integrity of the parental plasmid and the size of insert DNA will be determined by agarose gel electrophoresis. The next step will be to confirm that the isolated plasmid DNA does contain the gene for hemolytic activity observed in primary transformants. This will be accomplished by transforming the original S. cerevisiae strain, with each of the plasmid DNAs containing putative hemolysin-like gene, and analysis of transformants for hemolysis on human blood agar (Manns et al., 1994). After reconfirming that the putative clones harbors the Candida genes for hemolytic activity, plasmids will be transferred into E. coli for amplification. The high quality plasmid DNAs will be purified from E. coli and verified for the presence of insert DNA similar to the original insert in the primary transformants. We will then determine the complete nucleotide sequence of the selected clones. The derived nucleotide and amino acid sequences will be compared to DNA and protein data bases using Blast and Beauty software. Gish (1993); Worley et al. (1995). After confirming that the DNA fragment encodes the hemolytic activity, we will analyze the regulation of the gene and the physiological role of the gene product as described below:

1) NORTHERN BLOT. Northern blot analysis of total RNA will be performed to determine whether the hemolysin gene is regulated at the transcriptional level during the white-opaque transition, the bud-hypha transition and/or by environmental parameters such as temperature, pH and constituents of growth medium. The translational product of hemolysin gene will be analyzed by using specific epitope-tagged hemolysins.

2) LOSS OF FUNCTION. Gene knockout analysis will be done using the strain TS3.3, an ura3⁻ derivative of WO-1. The homozygous deletion mutants of the hemolysin gene(s) will be analyzed to determine the contribution of hemolysin genes in virulence in both skin and systemic animal models of infection. Kvaal et al. (1997).

3) MISEXPRESSION. If the expression of hemolytic activity is determined to be phase-specific (Kvaal et al., 1997), the misexpression of hemolysin genes will be analyzed.

4) DETERMINATION OF FUNCTIONAL DOMAINS. Protein domains required for hemolytic activity in strains harboring homozygous deletion for the gene will be identified by hemolytic activity on human blood agar. Initially, we will determine the domains or regions that are essential for hemolytic activity. Next, we will map critical amino acid residues by targeted point mutations of specific amino acid residues based on structure-functional studies of bacterial hemolysins.

5) PROTEIN MODIFICATION. First, we will establish whether hemolysin is modified by glycosylation. If the hemolysin is glycosylated, we will then determine whether glycosylation is required for hemolytic activity by using specific glycosylation inhibitors such as tunicamycin and specific anion transport inhibitors such as DIDS, SITS and BS (Watanabe et al., 1999). The role of identified carbohydrate-modified amino acids in hemolysis will be addressed by creating point mutations at specific amino acid residues.

### Example 4

### Search for HLP Homologs by “Mining” Nucleic Acid and Protein Databases

Known sequences from Candida HLPs may be compared to other known nucleotide sequences on various databases. For example, a search revealed three nucleic acids in Stanford's C. albicans genome database, con4-2646, con4-2740 and con4-2796, which were highly homologues to the HLP of C. glabrata. Identification of sequences on the basis of homology will then be followed by structural and functional characterization using the methods described above.

### Example 5

### Miscellaneous Methods of the Invention

Yeast isolates and maintenance. C. glabrata isolates may be obtained from any source, such as from blood of infected individuals. Isolates were typed as C. glabrata by sugar assimilation pattern and by hybridization to the C. glabrata-specific probes Cg6 and Cg12 (21). Clones were stored at room temperature on a YPD agar slant (2% glucose, 2% Bacto Peptone, 1% yeast extract, 2% agar, Difco Laboratories, Detroit, Mich.) in a capped tube. The switch phenotypes were propagated on YPD agar plates containing 1 mM CuSO₄ at 25° C. Each of the phenotypes may also be stored at −80° in glycerol.

Measurements of phenotypic switching. To assess the frequency of variant phenotypes in a clonal population of C. glabrata, cells from a single 3 day old colony exhibiting a homogenous colony phenotype were inoculated into YPD liquid medium containing 1 mM CuSO₄ and were grown at 25° C. for approximately 6 to 8 hr to a density of 5×10⁶ cells per ml. Cells then were diluted and were plated at a density of approximately 50 cells per agar plate. Plates were incubated at 25° C. for 5 days and the colony phenotypes were scored.

Growth kinetics. Cells from a 3 day old single colony exhibiting a homogeneous phenotype were inoculated into 10 ml of YPD liquid medium containing 1 mM CuSO₄ in a 30 ml test tube and incubated until the concentration reached 1×10⁷ cells per ml. Then 5×10⁶ cells were inoculated into a 250 ml Erlenmyer flask, containing 50 ml of fresh YPD liquid medium plus 1 mM CUSO₄, and were rotated at 25° C. for 48 hr. Samples were removed every 2 hr over a 48 hr period and were vortexed. The concentration of spheres was measured in a hemocytometer.

PCR amplification of C. glabrata and C. albicans HLP gene. PCR products were generated in 100 μl of a reaction mixture containing 10 mM Buffer B (Fischer Scientific, St. Louis, Mo.), 1.2 mM MgCl₂, 100 μM dNTP, 50 muM dNTP, 50 μM of the 5′ primer and 50 μM of the 3′ primer, and 2.5 units of Taq polymerase (Fischer Scientific). Conditions for PCR cycling included 40 cycles of denaturation at 92° C. for 1 min, annealing at 40° C. for 1.5 min, and extension at 68° C. for 1.5 min.

PCR products were gel-purified and used as template for generating radioactive probes. The C. glabrata HLP PCR product was obtained using primers SLF1-N5′ ATGTCATCGCAAAACCTCAAT3′ and SLF1-C5′ CTGCCTGCTAATTTCACCTTG3′. The PCR product was cloned in E. coli and sequenced in both directions using an ABI model 373A automatic sequencing system and fluorescent Big dye terminator chemistry (Perkin Elmer/Applied Biosystems Inc., Fostor city, CA). The alignment of nucleotide sequences and comparison with sequences in the databases were performed with the BLASTX-BEAUTY analysis program.

DNA fingerprinting and southern blot analysis. DNA fingerprinting was performed as described in Schmid et al. (1990) with complex DNA fingerprinting probes (Lockhart et al. (1997)). In brief, total genomic DNA from each of the C. glabrata switch phenotypes was prepared by the method of Scherer et al. (1987). Approximately 1 μg of total genomic DNA were digested with EcoRI (4 U/μg of DNA) and the resulting fragments electrophoresed at 35 V for 15 hr in a 0.65% (w/v) agarose gel. DNA was transferred by capillary blotting to Hybond N⁺nylon membrane (Amersham Parmacie Biotech, Buckinghamshire, England), hybridized with randomly primed [³²P] dCTP-labeled probe, and autoradiographed as described in Schmid et al. (1990). For Southern blot analyses performed for purposes other than DNA fingerprinting, DNA was digested with SalI, the digestion fragments were resolved in a 0.8% (w/v) agarose gel, and the Southern blots were hybridized with randomly primed [³²P] dCTP-labeled probes.

Slot blot and northern analysis of transcripts. Total cellular RNA was isolated by methods described in Srikantha et al. (1995) with the following modifications: pellets of 3×10⁸ washed cells from 3 day old colonies were frozen, mixed with an equal volume of acid-washed glass beads (400 μm diameter) and 450 μl of RNA extraction buffer from a RNAeasy Mini Kit (Qiagen Inc., Valencia, Calif.), and agitated with a bead beater device (Biospec Products, Bartlesville, Okla.). Two μg of total cell RNA were immobilized on a Zetabind Nylon membrane (CUNO, Inc., Meriden, Conn.) using the slot blot apparatus PR48 (Hoefer Pharmacia Biotech. Inc., San Francisco, Calif.), hybridized with randomly primed ³²P-labeled probe, and autoradioraphed. Hybridization intensities were compared by scanning the slot blots with the “Densitometry of Lanes” option of the DENDRON software package version 2.0 (SolltechInc., Iowa City, Iowa). To perform successive hybridizations of the slot blot with different probes, the previous probe was stripped using standard methods. Northern blot hybridization was performed according to methods described in Kvaal et al. (1997).

The description, specific examples, and data, while indicating exemplary embodiments, are given by way of illustration and are not intended to limit the present invention. Various changes and modifications within the present invention will become apparent to the skilled artisan from the disclosure, and thus are considered part of the invention.

### BIBLIOGRAPHY

Ausubel et al. (1989) Current Protocols in Molecular Biology, Green Publishing Associates and Wiley Interscience, N.Y.

Balan et al. (1997) J. Bacteriol. 179:7210-7218.

Bayer et al. (1979) Meth. Enzym. 62:308.

Bird (1988) Science 242:423-426.

Blaschke-Hellmessen et al. (1996) Mycoses 2: 39-43.

Boone et al. (1991) J. Bacteriol. 6859-6864.

Byrd et al. (1997) Curr. Microbiol. 34:149-154.

Campbell (1984) In Monoclonal Antibody Technology: Laboratory Techniques in Biochemistry and Molecular Biology, Elsevier Science Publishers, Amsterdam, The Netherlands.

Cole et al. (1985) In Monoclonal Antibodies and Cancer Therapy, Alan R. Liss, Inc., pp. 77-96.

Coligan et al. (1997 and periodic supplements) Current Protocols in Immunology, Greene Pub. Associates and Wiley-Interscience, New York, N.Y.

Colombo et al. (1999) Diagn. Microbiol. Infect. Dis. 34:281-286.

Current Protocols in Immunology, chapter 2, Coligan et al., eds., John Wiley & Sons (1991-92).

Engval et al. (1972) Immunol. 109:129.

Fairweather et al. (1983) Infect. Immun. 41:1112-1117.

Fattom et al. (1993) Infect. and Immun. 61:1023-1032.

Fidel et al. (1999) Clin. Microbiol. Rev. 12:80-96.

Fisher(1980) In Manual of Clinical Immunology, 2d ed., Rose and Friedman, eds., Amer. Soc. For Microbiology, Washington, D.C., Chap. 42.

Fortun et al. (1997) Eur. J. Clin. JMicrobiol. Infect. Dis. 16:314-318.

Fu et al. (1998) Infect. Immun. 66:1783-1786.

Furesz et al. (1997) Infect. Immun. 65:358-365.

Gaur et al (1997) Infect. Immun. 65:5289-5294.

Gibson et al. (1999) Pediat. Res. 45:626-634.

Gillum et al. (1984) Mol. Gen. Genet. 198:179-182.

Gish et al. (1993) Nat. Genet. 3:266-272.

Hacker et al. (1983) Infect. Immun. 42:57-63.

Haga et al. (1997) J. Vet. Med. Sci. 59:115-120.

Hampton et al. (1990), Serological Methods-A Laboratory Manual, APS Press, St Paul, Minn.

Hazen et al. (1995) Clin. Microbiol. Rev. 8:462-478.

Hellstein et al. (1993) J. Clin. Microbiol. 31:3190-3199).

Helms et al. (1977) J. Infect. Dis. 135:582-592.

Herbelin et al. (1995) J. Microb. Methods 21:163-171.

Hitchcock et al. (1993) Antimicrob. Agents Chemother. 37:1962-1965.

Hube et al. (1994) Mol. Microbiol. 14:87-99.

Huston et al. (1988) Proc. Natl. Acad. Sci. USA 85:5879-5883.

Jones et al. (1994) J. Clin. Microbiol. 32:2869-2870.

Kohler et al. (1975) Nature 256:495-497.

Kosbor et al. (1983) Immunology Today 4:72.

Kozbor et al. (1983) Immunology Today 4:72.

Kvaal et al. (1997) Infect. Immum. 65:4468-4475.

Kvaal et al. (1999) Infect. Immun., in press.

Lange et al. (1997) J. Biol. Chem. 272: 20884-20892.

Leao et al. (1995) Infect. Immun. 63:4301-4306.

Li et al. (1999) J. Bacteriol. 181:6019-6027.

Lockhart et al. (1997) Microbiology 143:3733-3746.

Lockhart et al. (1999) J. Dent. Res. 78:857-868.

Maddox et al. (1983) J. Exp. Med. 158:1211-1216.

Manns et al. (1994) Infect. Immunity 62:5154-5156.

Marichal et al. (1997) Antimicrob. Agents Chemother. 41:2229-2237.

Maslow et al. (1999) J. Clinical Microbiol. 37:445-446.

May et al. (1996) Infect. Immun. 64:2167-2171.

Morrison et al. (1984) Proc. Natl. Acad. Sci. 81:6851-6855.

Morrow et al. (1992) Mol. Cell. Biol. 12:2997-3005.

Morrow et al. (1993) Infect. Immun. 61:1823-1828.

Nabel et al. (1992) Hum. Gene Ther. 3:649-656.

Neuberger et al. (1984) Nature 312:604-608.

Nizet et al. (1997) Adv. Exp. Med. Biol. 418:627-630.

Odds (1997) Mycoses 40(Suppl. 2):9-12.

Pacheco-Rios et al. (1997) Arch. Med. Res. 28:229-232.

Parveen (1992) Microbios 70:67-70.

Pecorino et al. (1992) Current Biol. 2:30-32.

Pfaller (1996) Clin. Infect. Dis. 22(Suppl. 2):S89-94.

Rosenbluh et al. (1985) Mol. Gen. Genet. 200:500-502.

St. Groth et al. (1980) J. Immunol. Methods 35:1-21.

Sambrook et al. (1989) Molecular Cloning: A Laboratory Manual. CSHL Press, NY.

Scherer et al. (1987) J. Clin. Microbiol. 25:675-679.

Schiestl et al. (1989) Curr. Genet. 16:339-346.

Schmid et al. (1990) J. Clin. Microbiol. 28:1236-1243.

Serra et al. (1992) J. Biochem. Biophys. Methods 25:83-94.

Sobel et al. (1996) Semin. Dermatol. 15:17-28.

Soll et al. (1987) J. Clin. Microbiol. 25:1611-1622.

Soll et al. (1988) J. Clin. Microbiol. 26:1448-1459.

Soll (1992) Clin. Microbiol. Rev. 5:183-203.

Soll (1996) ASM News 62:415-420).

Spinillo et al. (1995) Obstet. Gynecol. 85:993-998.

Srikantha et al. (1995) Mol. Cell. Biol. 15:1797-1805.

Sternberger et al. (1970) J. Histochem. Cytochem. 18:315.

Takeda et al. (1985) Nature 314:452-454.

Thelestam et al. (1994) Methods Enzymol. 235:679-690.

Traub et al. (1996) Chemotherapy 42:118-132.

Tweten (1995) In Virulence Mechanisms of Bacterial Pathogens. Roth, J. A., ed. American Society for Microbiology, Washington D.C., pp. 207-229.

Vaitukaitis et al. (1971) J. Clin. Endocrinol. Metab. 33:988-991.

Verduyn Lunel et al. (1999) Diagn. Microbiol. Infect. Dis. 34:213-220.

Wanatabe et al. (1999) Microbiology 145:689-694.

Weir et al. (1986) In Handbook of Experimental Immunology 4th Ed., Blackwell Scientific Publications, Oxford, England, Chapter 10.

Welch (1995) In Virulence Mechanisms of Bacterial Pathogens. Roth, J. A.. ed. American Society for Microbiology, Washington D.C., pp 195-206.

White et al. (1993) J. Bacteriol. 175:6126-6133.

Wolff et al. (1990) Science 247:1465-1468.

Worley et al. (1995) Genome Res. 5:173-184.

What is claimed is:

1. A vaccine, comprising:
(i) an antigenic polypeptide comprising 10 contiguous amino acid residues of either of the proteins shown in SEQ ID NOS:2 or 4; or
(ii) a polynucleotide encoding an antigenic polypeptide comprising 10 contiguous amino acid residues of either of the proteins shown in SEQ ID NOS:2 or 4, wherein said polynucleotide is operably linked to a promoter capable of driving transcription in a host cell; and
(iii) a pharmaceutically acceptable adjuvant or carrier vehicle.

2. The vaccine of claim 1, wherein the vaccine comprises said polypeptide, and wherein said polypeptide comprises 12 contiguous amino acids of either of the proteins shown in SEQ ID NOS:2 or 4.

3. The vaccine of claim 1, wherein the vaccine comprises said polypeptide, and wherein said polypeptide comprises 30 contiguous amino acids of either of the proteins shown in SEQ ID NOS:2 or 4.

4. The vaccine of claim 1, wherein the vaccine comprises said polypeptide, and wherein said polypeptide comprises 40 contiguous amino acid residues of either of the proteins shown in SEQ ID NOS:2 or 4.

5. The vaccine of claim 1, wherein the vaccine comprises said polypeptide, and wherein said carrier vehicle is a protein that is covalently conjugated to said polypeptide.

6. The vaccine of claim 1, wherein the vaccine comprises said polynucleotide, and wherein said polynucleotide encodes a polypeptide comprising 10 contiguous amino acids of either of the proteins shown in SEQ ID NOS:2 or 4.

7. The vaccine of claim 1, wherein the vaccine comprises said polynucleotide, and wherein said polynucleotide encodes a polypeptide comprising 12 contiguous amino acids of either of the proteins shown in SEQ ID NOS:2 or 4.

8. The vaccine of claim 1, wherein the vaccine comprises said polynucleotide, and wherein said polynucleotide encodes a polypeptide comprising 30 contiguous amino acid residues of either of the proteins shown in SEQ ID NOS:2 or 4.

9. The vaccine of claim 1, wherein the vaccine comprises the protein shown in SEQ ID NO:2.

10. The vaccine of claim 1, wherein the vaccine comprises the protein shown in SEQ ID NO:4.

11. A method of diagnosing a virulent phenotype of Candida, comprising exposing a body fluid from an individual suspected of having a virulent phenotype of Candida with a detectably labeled antibody that is capable of binding an HLP of Candida.

12. A method of treatment of an individual infected with a virulent phenotype of Candida, comprising administration of a pharmaceutical composition that comprises an antibody that is capable of binding an HLP of Candida.

13. A method of inducing an immune response, comprising administering to an individual a pharmaceutical composition that comprises:
(i) an antigenic polypeptide comprising 10 contiguous amino acid residues of either of the proteins shown in SEQ ID NOS:2 or 4; or
(ii) a polynucleotide encoding an antigenic polypeptide comprising 10 contiguous amino acid residues of either of the proteins shown in SEQ ID NOS:2 or 4, wherein said polynucleotide is operably linked to a promoter capable of driving transcription in a host cell; and
(iii) a pharmaceutically acceptable adjuvant or carrier vehicle.

14. The method of claim 13, wherein the immune response is a cellular immune response.

15. An isolated polynucleotide that exhibits greater than about 80% sequence identity to polynucleotide sequence SEQ ID NO: 1.

16. The polynucleotide of claim 15, wherein said polynucleotide has the sequence shown in SEQ ID NO:1.

17. The polynucleotide of claim 15, wherein said polynucleotide codes for a polypeptide having the sequence shown in SEQ ID NO:2.

18. An isolated polypeptide encoded by the polynucleotide according to claim 15.

19. The polypeptide of claim 18, wherein said polypeptide has the sequence shown in SEQ ID NO:2.

20. An antigenic fragment of the polypeptide according to claim 19, wherein said fragment comprises at least 30 contiguous amino acids of the protein shown in SEQ ID NO:2.
